(19) United States
(12) Patent Application Publication
(10) Pub. No.: US 2006/0153892 A1
(43) Pub. Date: Jul. 13, 2006

(54) Protein-resistant articles

(76) Inventors: David Scott Porter, Blountville, TN (US); Craig Steven Sass, Kingsport, TN (US)

Correspondence Address: Louis N. Moreno;Eastman Chemical Company, P.O. Box 511, Kingsport, TN, 37662-5075, US

(21) Appl. No.: 11/313,607

(22) Filed: Dec. 20, 2005

Related U.S. Application Data
(60) Provisional application No. 60/642,622, filed on Jan. 10, 2005.

(51) Int. Cl. B05D 3/02; A61F 13/00

(52) U.S. Cl. 424/422; 427/2.26

(57) ABSTRACT

This invention relates to the reduction of interaction of biological systems with foreign substrates. Some applications require that the contact of a biological fluid with a foreign surface, such as a container for that fluid, have minimal interactions with that surface. Because the adsorption of proteins at surfaces mediates much biological response, minimization of protein adsorption is a goal to improve the overall biocompatibility of materials. Described herein is a technique in which silicone coatings that are cured by UV light are applied to surfaces. The silicone surface minimizes the adsorption of proteins, and because the coatings are cured by UV light, the cure is rapid and suitable for high-speed application. Additionally, because the UV treatment causes little or no heating of the substrate, this technique is especially suitable for temperature-sensitive substrates, such as those sometimes used for medical devices. The use of UV curing also allows patterned substrates to be developed.

### CROSS-REFERENCE TO RELATED APPLICATION

This application claims the benefit of U.S. Provisional Patent Application No. 60/642,622, filed on Jan. 10, 2005; the entire content of which is hereby incorporated by reference.

### FIELD OF THE INVENTION

The invention generally relates to protein-resistant articles. More particularly, the invention relates to articles comprising a UV-cured silicone polymer coating composition, and a method for reducing interaction between an article and a biological fluid or system.

### BACKGROUND OF THE INVENTION

This invention pertains to the improvement of protein resistance and biocompatibility of articles that come into contact with biological systems through the application of biocompatible coatings. These coatings have uses in many different areas in which the adsorption of proteins may be problematic, such as diagnostic tests in which quantification of the amount of proteins in a sample may be complicated by adsorption of proteins at the surface, as well as operations in which the buildup of proteins can prevent proper operation, such as filtration apparatus. Additionally, the importance of biocompatible articles arises in part from their utility in medical devices. The term “medical device,” as used herein, describes an apparatus that is used in the diagnosis or treatment of a disease and that come into contact with biological materials from animals, humans or plants, including tissue, blood, or other biological fluids. The term ‘biocompatible’ is used herein to describe the effect of substantially reducing by greater than about 50%, preferably greater than about 80%, more preferably by greater than about 90% or minimizing or eliminating completely the interaction between a biological system and the introduced foreign surface. The term “protein-resistant” is used herein to describe a reduced tendency to adsorb protein compared to an uncoated surface or article.

Though a material used for a particular application might have low reactivity, low levels of extractable substances, and/or be otherwise inert, biological systems may have adverse reactions to the introduction of such a foreign surface. This is due to the interaction of proteins with the surface. It is accepted that the first observable event to occur when a foreign surface contacts a biological system is the adsorption of proteins, and this adsorption can dictate the type and extent of the response to that surface. (J. D. Andrade and V. Hlady, Protein Adsorption and Materials Biocompatibility: A Tutorial Review and Suggested Hypotheses, in Advances in Polymer Science, 79, (1986), p. 3; L. Vroman and A. L. Adams, Journal of Biomedical Materials Research, 3, (1969), p.43.)

One approach to overcoming any negative effects associated with the contact of a surface with a biological system is to form the entire article out of a biocompatible material. While several materials have been identified as biocompatible, these materials may not possess all of the other necessary properties to be successfully employed. The particular needs of an application may dictate that a particular article be formed of materials with specific characteristics, examples of which are physical properties such as stiffness or optical clarity.

To satisfy both requirements, the present inventors have adopted the approach of modifying the surface of a material having suitable bulk properties to improve biocompatibility. In particular, the present inventors have adopted the approach of applying a coated layer of a more biocompatible material over another material with the appropriate physical properties.

The present invention is particularly directed to an ultraviolet light (UV)-curable, silicone-based coating which improves protein resistance and biocompatibility, may be coated on various substrates, and overcomes several difficulties identified in previously disclosed methods.

### SUMMARY OF THE INVENTION

In one aspect, the invention provides a protein-resistant medical device that comprises a UV-cured silicone polymer coating on at least a portion of the surface thereof.

In another aspect, the invention provides a method for reducing interaction between a medical device and a biological fluid or system. The method comprises coating at least a portion of a surface of the device with a UV-curable silicone polymer composition and exposing at least a portion of the silicone polymer composition to ultraviolet light to cure the composition.

The use of a UV-curable silicone polymer coating composition allows for rapid curing, low-temperature curing for temperature-sensitive substrates, as well as patterning of the coated substrate.

### DETAILED DESCRIPTION OF THE INVENTION

The present invention may be understood more readily by reference to the following detailed description of preferred embodiments of the invention and the Examples included therein.

Unless otherwise indicated, all numbers expressing quantities of ingredients, properties such as thickness, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, each numerical parameter should at least be construed in light of the number of reported significant digits and by applying ordinary rounding techniques. Further, the ranges stated in this disclosure and the claims are intended to include the entire range specifically and not just the endpoint(s). For example, a range stated to be 0 to 10 is intended to disclose all whole numbers between 0 and 10 such as, for example 1, 2, 3, 4, etc., all fractional numbers between 0 and 10, for example 1.5, 2.3, 4.57, 6.1113, etc., and the endpoints 0 and 10.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

As used in the specification and the appended claims, the singular forms “a,” “an” and “the” include their plural referents unless the context clearly dictates otherwise. For example, reference a “silicone polymer coating,” or a “curing agent,” is intended to include the processing or making of a plurality of polymer coatings, or curing agents. References to a composition containing or including “an” ingredient or “a” polymer is intended to include other ingredients or other polymers, respectively, in addition to the one named.

By “comprising” or “containing” or “including” we mean that at least the named compound, element, particle, or method step, etc., is present in the composition or article or method, but does not exclude the presence of other compounds, catalysts, materials, particles, method steps, etc, even if the other such compounds, material, particles, method steps, etc., have the same function as what is named, unless expressly excluded in the claims.

It is also to be understood that the mention of one or more method steps does not preclude the presence of additional method steps before or after the combined recited steps or intervening method steps between those steps expressly identified. Moreover, the lettering of process steps or ingredients is a convenient means for identifying discrete activities or ingredients and the recited lettering can be arranged in any sequence, unless otherwise indicated.

In one embodiment the present invention relates to medical devices, such as lab-ware and components of diagnostic test kits, that may come into contact with biological fluids or biological systems and that have a reduced interaction with that biological fluid or system. Medical devices include, but are not limited to, diagnostic equipment, such tubes, bottles, bags, and other containers; fluid handling apparatus, such as intravenous (IV) systems including needles and hubs, cannulae, tubing, connectors and other fixtures; blood treatment and dialysis equipment, including dialyzers, filters, and oxygenators; anesthesia and respiratory therapy equipment, such as masks and tubing; drug delivery and packaging supplies, such as syringes, tubing, transdermal patches, inhalers, bags and bottles; catheters, tubes, and endoscopy equipment; and labware, including dishes, vials, plates and cell culture equipment. The devices comprise a UV-cured silicone polymer coating, which is applied to a surface of the device so as to reduce the response of the biological fluid or system in contact with the device. The resultant devices possess a thin, adherent coating of silicone polymer which gives biocompatibility. By using a coating, the advantageous properties of the substrate material may be obtained, which may include stiffness, clarity, favorable economics or other desirable properties. In another embodiment the present invention relates to a method of reducing the interaction between a medical device and a biological fluid or system, the method comprising coating at least a portion of a surface of the device with a UV-curable silicone polymer composition and exposing at least a portion of the silicone polymer composition to ultraviolet light to cure the composition.

The UV-curable silicone polymer composition can be applied to nearly any substrate known in the art for use in medical devices. Such substrates include, for example, plastics, elastomers, metals and the like. Specific materials include polyvinylchlorides (PVC), polycarbonates (PC), polyurethanes (PU), polypropylenes (PP), polyethylenes (PE), silicones, polyesters, cellulose acetates, polymethylmethacrylates (PMMA), hydroxyethylmethacrylates, N-vinyl pyrrolidones, fluorinated polymers such as polytetrafluoroethylene, polyamides, polystyrenes, copolymers or mixtures of the above polymers and medical grade metals such as steel or titanium.

Examples of UV-curable silicone polymers that can be used in the coating composition of the invention include polymers composed of at least 50 mole% dimethyl siloxane repeat units. Other suitable UV-curable silicone polymers are known in the art such as those mentioned in U.S. Pat. Nos. 4,576,999; 4,279,717; 4,421,904; 4,547,431; 4,576,999; and 4,977,198; the entire contents of which are hereby incorporated by reference.

The coating composition may be applied by any number of methods, including but not limited to spraying, dipping, printing, or flow-coating. Other methods of application known in the art are also to be considered within the scope of this invention. Further, the polymer may be used in solution or emulsified to reduce its viscosity for application. A diluent, if employed, may be allowed to evaporate, and this evaporation may be facilitated by applying energy via heat or radiation. Optionally, evaporation of all or part of the solvent may be accomplished after a curing operation.

Any solvent that is capable of dissolving or substantially dissolving the silicone polymer such that its viscosity is reduced for application may be used. Examples of such solvents include aliphatic or aromatic hydrocarbons, such as toluene and cyclohexane; volative silicones such as cyclomethicone; chlorinated hydrocarbons; and esters (see, e.g., Polymer Handbook, Brandup and Immergut, Eds., 2nd edition, page IV-253 (1975)). In addition, the viscosity of the coatings could be decreased through emulsification, or lowering the molecular weight of the silicones.

The silicone polymer coating composition may further include one or more UV curing agents to help facilitate curing of the composition. Suitable UV curing agents may be obtained commercially from vendors of the UV-curable silicone polymers such as General Electric Co. Suitable UV curing agents are also known in the art such as in U.S. Pat. Nos. 4,576,999; 4,279,717; 4,421,904; 4,547,431; 4,576,999; and 4,977,198.

Curing of the coating may be achieved by exposure to UV radiation, which may be produced by any convenient means. The curing time depends on a number of factors including the precise polymer composition and the desired degree of cross-linking. Preferably, the curing time is less than 5 seconds.

The finished coating may have a range of thicknesses, from several nanometers up to several millimeters, preferably from 0.1 to 100 micrometers. Similarly, the substrate thickness may vary, from about 0.001 millimeters to about 100 millimeters, preferably from about 0.01 millimeters to about 10 millimeters.

The ability to cure using ultraviolet light rather than a thermally cured polysiloxane is desirable in areas in which the substrate might be sensitive to elevated temperatures. For devices used in medical applications, this is a common concern as not all materials can withstand elevated temperature in procedures such as steam sterilization. For temperature-sensitive substrates, other sterilization methods can be used that do not involve the application of heat, such as gamma irradiation or ethylene oxide treatment. The use of UV curing polysiloxanes according to the present invention allows these same temperature-sensitive substrates to be made biocompatible. By “temperature-sensitive substrates,” it is meant substrates that can irreversibly change their characteristics (such as dimensions, shape, color, brittleness, crystallinity, etc.) at elevated temperatures typically employed in medical or diagnostic applications. Examples of such substrates include polymers having relatively low softening, melting, or glass transition temperature points.

Additionally, by crosslinking the coating using UV radiation, patterned surfaces may be formed. In this way, selective areas may be made to resist protein adsorption, while other areas may be receptive to protein adsorption. By exposing selected areas to UV light, the non-exposed, non-crosslinked areas may be subsequently removed by various techniques, such as solvent washing. This could produce patterned areas of relatively low and high protein binding, for analytical tests and other applications.

An embodiment of the present invention is further illustrated by the following example. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims. Moreover, all patents, patent application (published and unpublished, foreign or domestic), literature references or other publications noted above are incorporated herein by reference for any disclosure pertinent to the practice of this invention.

### EXAMPLE

A coating composition was formed by mixing an epoxy-functional polysiloxane with a UV curing additive. The silicone used was available as General Electric 9300 silicone release agent, and the UV curing agent used was General Electric UV9380c. 50 grams of the silicone coating was stirred with 1 gram of the UV curing agent until uniformly mixed. This coating was applied to an amorphous extruded polyethylene terephthalate film. The coated film was passed into a UV curing apparatus (American Ultraviolet mini conveyorized UV cure system) at 50 feet per minute at a power density setting of 200 watts per inch.

Additionally, extruded films of polyethylene, polystyrene, PCTG, PETG and cellulose acetate were examined uncoated.

Biocompatibility was determined by measuring the adsorption of protein from solution. The samples were first sonicated in water for 10 minutes, followed by pretreatment in phosphate buffer for 24 hours. The samples were then immersed for 30 minutes in a 0.1mg/mL solution of bovine fibrinogen, removed and immersed for 30 minutes in clean phosphate buffer solution. The samples were removed from the buffer, rinsed with deionized water, and dried in vacuum for 24 hours. These samples were examined for surface atomic composition using X-ray photoelectron spectroscopy (XPS). Because the fibrinogen contains nitrogen and the substrate polymers do not, the quantity of nitrogen detected at the surface is proportional to the propensity for the surface to accumulate or adsorb proteins. It is this adsorption of proteins at the surface that controls the interaction of a biological system with a surface.

| Substrate | % surface nitrogen |
|---|---|
| PET | 5.3 |
| Copolyester “PETG” | 6.6 |
| Copolyester “PCTG” | 5.6 |
| Cellulose Acetate | 4.7 |
| Polypropylene | 3.1 |
| Silicone-coated PET | 0.3 |

As seen from the results above, coatings of UV-cured silicone materials on polymer substrates can substantially decrease the amount of fibrinogen adsorbed onto surfaces as evidenced by a lower indicated % surface nitrogen.

We claim:

1. A protein-resistant medical device, said device comprising a UV-cured silicone polymer coating on at least a portion of the surface thereof.

2. The device of claim 1, wherein the coating comprises an epoxy-functional polysiloxane and a UV curing agent.

3. The device of claim 1, further comprising a patterned surface defined by the coating.

4. A protein-resistant device, said device comprising a temperature-sensitive substrate and a UV-cured silicone polymer coating on at least a portion of the substrate.

5. The device of claim 4, wherein the coating comprises an epoxy-functional polysiloxane and a UV curing agent.

6. A method for reducing interaction between a medical device and a biological fluid or system, said method comprising:
coating at least a portion of a surface of the device with a UV-curable silicone polymer composition; and
exposing at least a portion of said UV-curable silicone polymer composition to ultraviolet light to cure the composition.

7. The method of claim 6, wherein the silicone polymer composition comprises an epoxy-functional polysiloxane and a UV curing agent.

8. The method of claim 6, further comprising:
removing any uncured silicone polymer composition from the surface of the device to produce a patterned surface comprising areas of relatively low protein binding and relatively high protein binding.

9. The method of claim 6, wherein the curing time is 5 seconds or less.
